(19) United States
(12) Patent Application Publication
(10) Pub. No.: US 2010/0210611 A1
(43) Pub. Date: Aug. 19, 2010

(54) COMBINATION THERAPY

(76) Inventor: Roch Thibert, Mont-Royal (CA)

Correspondence Address: MERCK, P O BOX 2000, RAHWAY, NJ, 07065-0907, US

(21) Appl. No.: 12/738,946

(22) PCT Filed: Oct. 23, 2008
(86) PCT No.: PCT/CA08/01874

§ 371 Date: Apr. 20, 2010

(30) Foreign Application Priority Data
Oct. 25, 2007 (US) 61/000327

(51) Int. Cl. A61K 31/58; A61K 31/444; A61P 11/06

(52) U.S. Cl. 514/171; 514/300

(57) ABSTRACT

The present invention provides inhalation compositions comprising montelukast acid and a second active agent selected from a PDE4 inhibitor and an inhaled corticosteroid. Also provided is a method for the treatment of respiratory disorders such as asthma using such compositions.

### BACKGROUND OF THE INVENTION

Major drug classes commonly used in the treatment of chronic asthma include bronchodilators (β-agonists, anticholinergics), corticosteroids, mast cell stabilizers, leukotriene modifiers, and methylxanthines. Most of these therapies are administered to the patient by the inhaled route, either in aerosolized or powdered form, and some recently introduced inhalation products are combination of active agents from different therapeutic classes; ADVAIR and SYMBICORT are both combinations of a corticosteroid and a long-acting β-agonist. Montelukast sodium, a leukotriene antagonist, is the active agent in SINGULAIR®, a drug product approved for the treatment of asthma and allergic rhinitis. While montelukast is available as tablets and granules for oral administration, the use of the active moiety in inhalation has not been previously explored.

### SUMMARY OF THE INVENTION

The present invention provides medicinal preparations comprising montelukast acid and a second active agent in a combined preparation for administration by inhalation. Also provided is a method for the treatment of asthma using such inhalable combinations.

### BRIEF DESCRIPTION OF THE DRAWINGS

FIG. 1 shows the X-ray powder diffraction pattern for crystalline montelukast acid.

### DETAILED DESCRIPTION OF THE INVENTION

The present invention provides a medicinal preparation comprising montelukast acid and a second active agent selected from a PDE-4 inhibitor and an inhaled corticosteroid as a combined preparation for simultaneous, sequential or separate administration by inhalation.

In one aspect the medicinal preparation comprises montelukast acid and the PDE-4 inhibitor N-cyclopropyl-1-[3-(1-oxido-3-pyridinylethynyl)phenyl]-1,4-dihydro[1,8]-naphthyridin-4-one-3-carboxamide (hereinafter referred to as Compound X).

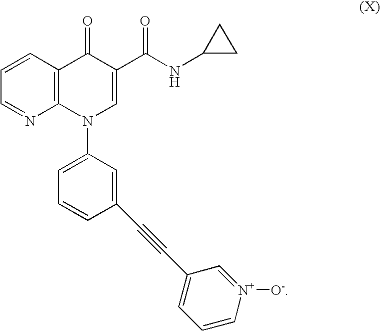

In another aspect the medicinal preparation comprises montelukast acid and an inhaled corticosteroid. In one embodiment the inhaled corticosteroid is selected from mometasone furoate and ciclesonide.

In another aspect the medicinal preparation comprises montelukast acid, and a second active agent selected from a PDE-4 inhibitor and an inhaled corticosteroid, wherein at least 95 percent of said montelukast acid and said second active agent having a particle size of 10 micron or less. The medicinal preparation of the present invention may be dispensed using either pressurized metered dose inhalers (pMDIs) or dry powder inhalers (DPIs).

The present invention further provides for the use of montelukast acid and a second active agent selected from a PDE-4 inhibitor and an inhaled corticosteroid in the manufacture of a combined preparation for administration by inhalation for the treatment of respiratory disorders.

The present invention additionally provides for a method for the treatment of respiratory disorders which comprises the simultaneous, sequential or separate administration by inhalation to a patient in need thereof a therapeutically effective amount of montelukast acid and a therapeutically effective amount of a second active agent selected from a PDE-4 inhibitor and an inhaled corticosteroid.

The present invention further provides for a dry powder inhaler containing the medicinal preparation described above. The present invention further provides for a metered dose inhaler containing the medicinal preparation described above.

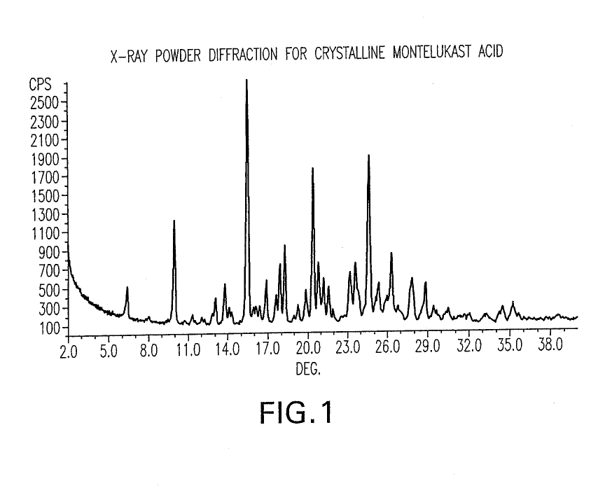

As used herein, the term “montelukast acid” refers to crystalline montelukast acid having X-ray powder diffraction pattern substantially as shown in FIG. 1. The term “PDE-4 inhibitors” refers to compounds which inhibit the actions of the phosphodiesterase-4 enzyme, and includes, without limitation, cilomilast, roflumilast, and Compound X. Compound X, uses of the compound and methods of making same are disclosed in WO 03/018579, published Mar. 6, 2003 and WO2004/048377, published Jun. 10, 2004. “Inhaled corticosteroids” include, but are not limited to, dexamethasone, fluticasone propionate, beclomethasone, budesonide, flunisolide, mometasone furoate, ciclesonide, and triamcinolone acetonide, as well as derivatives of each of the named inhaled corticosteroids; preferred inhaled corticosteroids are mometasone furoate, which is the active agent in the product ASMANEX, and ciclesonide, which is the active agent in the product ALVESCO.

The weight ratio of montelukast acid and the second active agent of the present preparation is in the range of about 10:1 to about 1:10. In a preparation where Compound X is the second active agent, the ratio is generally within the range of about 5:1 and about 1:5. In preparations where mometasone furoate is the second active agent, the ratio is generally within the range of about 5:1 and 1:5. In preparations where ciclesonide is the second active agent, the ratio is generally within the range of about 10:1 and about 1:1.

In one embodiment the medicinal preparation is adapted for use with a pressurized metered dose inhaler which releases a metered dose of medicine upon each actuation. The formulation for pMDIs can be in the form of solutions or suspensions in halogenated hydrocarbon propellants. The type of propellant being used in pMDIs is being shifted to hydrofluoroalkanes (HFAs), also known as hydrofluorocarbons (HFCs) as the use of chlorofluorocarbons (known also as Freons or CFCs) is being phased out. In particular, 1,1,1,2-tetrafluoroethane (HFA 134a) and 1,1,1,2,3,3,3-heptafluoropropane (HFA 227) are used in several currently marketed pharmaceutical inhalation products. The composition may include other pharmaceutically acceptable excipients for inhalation use such as ethanol, oleic acid, polyvinylpyrrolidone and the like.

Pressurized MDIs typically have two components. Firstly, there is a canister component in which the drug particles are stored under pressure in a suspension or solution form. Secondly, there is a receptacle component used to hold and actuate the canister. Typically, a canister will contain multiple doses of the formulation, although it is possible to have single dose canisters as well. The canister component typically includes a valve outlet from which the contents of the canister can be discharged. Aerosol medication is dispensed from the pMDI by applying a force on the canister component to push it into the receptacle component thereby opening the valve outlet and causing the medication particles to be conveyed from the valve outlet through the receptacle component and discharged from an outlet of the receptacle. Upon discharge from the canister, the medication particles are “atomized”, forming an aerosol. It is intended that the patient coordinate the discharge of aerosolized medication with his or her inhalation, so that the medication particles are entrained in the patient's aspiratory flow and conveyed to the lungs. Typically, pMDIs use propellants to pressurize the contents of the canister and to propel the medication particles out of the outlet of the receptacle component. In pMDIs, the formulation is provided in a liquid or suspension form, and resides within the container along with the propellant. The propellant can take a variety of forms. For example, the propellant can comprise a compressed gas or liquefied gas.

In another embodiment the medicinal preparation is adapted for use with a dry powder inhaler. The inhalation composition suitable for use in DPIs typically comprises particles of the active ingredient and particles of a pharmaceutically acceptable carrier. The particle size of the active material may vary from about 0.1 μm to about 10 μm; however, for effective delivery to the distal lung, at least 95 percent of the active agents particles are 5 μm or smaller. Each of the active agent can be present in a concentration of 0.01-99%. Typically however, each of the active agents is present in a concentration of about 0.05 to 50%, more typically about 0.2-20% of the total weight of the composition.

As noted above, in addition to the active ingredients, the inhalable powder preferably includes pharmaceutically acceptable carrier, which may be composed of any pharmacologically inert material or combination of materials which is acceptable for inhalation. Advantageously, the carrier particles are composed of one or more crystalline sugars; the carrier particles may be composed of one or more sugar alcohols or polyols. Preferably, the carrier particles are particles of dextrose or lactose, especially lactose. In embodiments of the present invention which utilize conventional dry powder inhalers, such as the Rotohaler, Diskhaler, and Turbohaler, the particle size of the carrier particles may range from about 10 microns to about 1000 microns. In certain of these embodiments, the particle size of the carrier particles may range from about 20 microns to about 120 microns. In certain other embodiments, the size of at least 90% by weight of the carrier particles is less than 1000 microns and preferably lies between 60 microns and 1000 microns. The relatively large size of these carrier particles gives good flow and entrainment characteristics. Where present, the amount of carrier particles will generally be up to 95%, for example, up to 90%, advantageously up to 80% and preferably up to 50% by weight based on the total weight of the powder. The amount of any fine excipient material, if present, may be up to 50% and advantageously up to 30%, especially up to 20%, by weight, based on the total weight of the powder.

The present invention in one embodiment provides a composition for use in dry powder inhaler, which comprises montelukast acid and Compound X, and lactose for inhalation as a carrier, wherein said composition is adapted for simultaneous, sequential or separate administration of the active agents. The weight ratio of lactose to montelukast acid is from about 1:1 to about 30:1, and to Compound X is from about 20:1 to about 30:1. In one instance the weight ratio of lactose to montelukast acid is about 2:1 to about 25:1, and to Compound X is about 20:1 to about 25:1.

The present invention in one embodiment provides a composition for use in dry powder inhaler, which comprises montelukast acid and an inhaled corticosteroid, and lactose for inhalation as a carrier, wherein said composition is adapted for simultaneous, sequential or separate administration of the active agents. In such compositions the weight ratio of lactose to montelukast acid is generally from about 1:1 to about 30:1. In a composition where the inhaled corticosteroid is mometasone furoate, the weight ratio of lactose to mometasone furoate is from about 130:1 to about 4:1, and in one embodiment the ratio is from about 124:1 to about 60:1. In a composition where the inhaled corticosteroid is ciclesonide, the weight ratio of lactose to ciclesonide is about 350:1 to about 100:1.

The powder may also contain fine particles of an excipient material, which may for example be a material such as one of those mentioned above as being suitable for use as a carrier material, especially a crystalline sugar such as dextrose or lactose. The fine excipient material may be of the same or a different material from the carrier particles, where both are present. The particle size of the fine excipient material will generally not exceed 30 μm, and preferably does not exceed 20 μm. In some circumstances, for example, where any carrier particles and/or any fine excipient material present is of a material itself capable of inducing a sensation in the oropharyngeal region, the carrier particles and/or the fine excipient material can constitute the indicator material. For example, the carrier particles and/or any fine particle excipient may comprise mannitol.

The formulations described herein may also include one or more additives, in an amount from about 0.1% to about 10% by weight, and preferably from about 0.15% to 5%, most preferably from about 0.5% to about 2%. Additives may include, for example, magnesium stearate, leucine, lecithin, and sodium stearyl fumarate. When the additive is micronized leucine or lecithin, it is preferably provided in an amount from about 0.1% to about 10% by weight, preferably about 0.5% to about 5%, preferably about 2%, of micronized leucine. Preferably, at least 95% by weight of the micronized leucine has a particle diameter of less than 150 microns, preferably less than 100 microns, and most preferably less than 50 microns. Preferably, the mass median diameter of the micronized leucine is less than 10 microns.

If magnesium stearate or sodium stearyl fumarate is used as the additive, it is preferably provided in an amount from about 0.05% to about 5%, preferably from about 0.15% to about 2%, most preferably from about 0.25 to about 0.5%.

Where reference is made to particle size of particles of the powder, it is to be understood, unless indicated to the contrary, that the particle size is the volume weighted particle size. The particle size may be calculated by a laser diffraction method. Where the particle also includes an indicator material on the surface of the particle, advantageously the particle size of the coated particles is also within the preferred size ranges indicated for the uncoated particles.

The dry powder pharmaceutical compositions in accordance with this invention may be prepared using standard methods. The pharmaceutically active agents, carrier particles, and other excipients, if any, may be intimately mixed using any suitable blending apparatus, such as a tumbling mixer. The particular components of the formulation can be admixed in any order. Pre-mixing of particular components may be found to be advantageous in certain circumstances. The powder mixture is then used to fill capsules, blisters, reservoirs, or other storage devices for use in conjunction with dry powder inhalers.

In a dry powder inhaler, the dose to be administered is stored in the form of a non-pressurized dry powder and, on actuation of the inhaler; the particles of the powder are inhaled by the patient. DPIs can be unit-dose devices in which the powder is contained in individual capsules, multiple-unit dose in which multiple capsules or blisters are used, and reservoir devices in which the powder is metered at dosing time from a storage container. Dry powder inhalers can be “passive” devices in which the patient's breath is used to disperse the powder for delivery to the lungs, or “active” devices in which a mechanism other than breath actuation is used to disperse the powder. Examples of “passive” dry powder inhaler devices include the Spinhaler, Handihaler, Rotahaler, Diskhaler, Diskus, Turbuhaler, Clickhaler, etc. Examples of active inhalers include Nektar Pulmonary Inhaler (Nektar Therapeutics), Vectura Limited's Aspirair™ device, Microdose DPI (MicroDose), and Oriel DPI (Oriel). It should be appreciated, however, that the compositions of the present invention can be administered with either passive or active inhaler devices.

Another aspect of the present invention provides a method for the treatment of respiratory disorders which comprises the simultaneous, sequential, or separate administration by inhalation to a patient in need thereof a therapeutically effective amount of montelukast acid and a therapeutically effective amount of a second active agent selected from a PDE-4 inhibitor and an inhaled corticosteroid. In one embodiment the respiratory disorder is asthma. In another embodiment, the second active agent is mometasone furoate or ciclesonide and the respiratory disorder is asthma.

The preparation of the present invention may be used in the treatment of asthma, COPD, pulmonary fibrosis, cough and other lung pathologies. The dosages for the individual active agents are typically those when used as a single therapeutic agent; the combination of active agents may be synergistic resulting in lower dose for one or both of the active agents or in reduced frequency of administration. The oral dose of montelukast sodium for the treatment of asthma ranges from 4 mg once daily for pediatric patients to 10 mg once daily for adult patients. The dose of montelukast acid for treating asthma using the inhalation composition of the present invention may be the same or less than the oral dose and may range from about 100 μg to about 10 mg per day; in one embodiment the dose is from about 200 μg to about 5 mg per day; in another embodiment the dose is from about 250 μg to about 2 mg per day; in another embodiment, the dose is from about 600 μg to about 4 mg per day. The dosage for compound X is disclosed in WO 03/018579 and WO2004/048377. The dosage for mometasone furoate may be from about 220 mcg to about 880 mcg per day, and may be lower when used in combination with montelukast acid; guidance for the dose range of mometasone furoate may be found in U.S. Pat. No. 5,889,015. The dosage for ciclesonide may be from about 80 to about 160 mcg per day, and may be lower when used in combination with montelukast acid; range of dosage for ciclesonide may be found in PCT Published Application WO2005025578. The combination of the present invention may be administered once, twice or thrice per day, and each administration may require more than one puff depending on the formulation, device, and dose to be administered. The inhaled dose for treating COPD, pulmonary fibrosis, cough and other leukotriene-mediated pulmonary pathologies is similar to that used for asthma.

The following examples are presented to illustrate the invention and are not meant to limit the scope of the claims in any manner.

### EXAMPLE 1

### Montelukast Acid

### Preparation of Crystalline Montelukast Acid

Acetic acid (124 ml, 0.247 mol) was added to a 6 L Erlenmeyer flask which had been charged with montelukast sodium (100 g, 0.165 mol), toluene (2.4 L) and water (1.6 L). The flask was protected from light with aluminum foil and the mixture was stirred with a magnetic stir bar for 10 min. The aqueous layer was separated and the organic layer washed with water (3×1L). The organic layer was stirred in the dark for 18 h. The resulting precipitate was filtered and dried under vacuum at 35° C. to afford 62 g of a yellow solid. A second crop of 14 g was recovered by extracting the aqueous washes with toluene (1×800 mL). The first crop was jet milled to afford 53 g of material with predominantly irregular crystals of <5 microns, with some rectangles as large as 8×5 microns. The material was 99.8% pure by HPLC.

### Preparation of Dry Powder Inhalation (DPI) Formulations

Two formulations were prepared in the same manner by blending in a Turbula tumbling mixer (Type T2F) for 15 minutes at 32 rpm inhalation grade lactose and montelukast acid. Two blends containing 4% montelukast acid were manufactured, one at a scale of 1 g and one at a scale of 10 g. One blend containing 20% montelukast acid was manufactured at a scale of 10 g. Capsules were filled with 25 mg of blend, equivalent to 1 mg of drug for the 4% w/w drug loading and 5 mg for the 20% w/w drug loading. The formulations are described in Table 1.

TABLE 1
DPI formulations with 4% and 20% drug loadings

| | | Formulation | | |
|---|---|---|---|---|
| Ingredient | Function | 4% w/w | 4% w/w | 20% w/w |
| Lactose for inhalation | Carrier | 96 | 96 | 80 |
| Montelukast Acid | API* | 4 | 4 | 20 |
| Batch size (g) | — | 1 | 10 | 10 |
| Shot weight (mg) | — | 25 | 25 | 25 |
| Capsule size | — | 2 | 2 | 2 |
| Dose (mg) | — | 1 | 1 | 5 |

*API = active pharmaceutical ingredient

### Blend Uniformity

To assess blend uniformity, capsules from each blend were opened and rinsed with methanol. The solution was sonicated for 5 minutes at room temperature, centrifuged at 3000 rpm for 15 minutes then assayed using a UV-VIS spectrophotometer at a wavelength of 346 nm.

The blend uniformity results for the blends of 4% w/w and 20% w/w drug loadings are summarized in Table 2. The results show that all blends were uniform with the amount of drug content within ±10% of the nominal doses. Blend uniformity results for the 4% w/w blends were independent of the batch size prepared.

TABLE 2
Individual capsule assay for blends I, II and III.

| | Drug | | | |
|---|---|---|---|---|
| | load | Batch size | Capsule | Mass of Montelukast Acid |
| Blend ID | (% w/w) | (g) | number | recovered in capsule (mg) |
| I | 4 | 1 | 1 | 0.91 |
| | | | 2 | 0.99 |
| | | | 3 | 1.02 |
| II | 4 | 10 | A | 0.98 |
| | | | B | 0.96 |
| III | 20 | 10 | A | 4.86 |
| | | | B | 5.35 |

### Dose Uniformity

Dose uniformity was determined using Apparatus B (Dosage Unit Sampling Apparatus—DUSA) at a flow rate of not more than 100 L/min (test described in USP <601>). The current USP recommends selecting a flow rate that creates a pressure drop of 4 kPa across the inhaler. With the Spinhaler®, a 4 kPa pressure drop and a flow rate of 100 L/min could not be achieved. Based on the recommendations of Byron, et al [Hindle and Byron, Int. J. Pharmaceutics, 116 (1995):169-177], a flow rate of 100 L/min should be selected since the Spinhaler® is a low resistance device.

During the DUSA studies, the first experiment performed with Spinhaler® succeeded to achieve a 4 kPa pressure drop, and a flow rate at approximately 100 L/min with a ratio P3/P2<0.5 (Table 3). For all subsequent experiments, a flow rate of only approximately 55 L/min could be achieved, however, with a ratio P3/P2>0.5. In order to ensure that subsequent experiments performed have a flow rate less than 100 L/min, a flow meter was connected to the intake port of the flow controller and the air flow rate was adjusted to approximately 100 L/min. By adjusting the air flow rate as described above, the pump was able to produce a sonic air flow across the DUSA with a ratio of P3/P2<0.5. After a shot had been delivered, all pieces of the DUSA including the mouthpiece adapter were rinsed with solvent, diluted to suitable volumes, sonicated and centrifuged. To determine the amount of drug retained in the inhaler, all pieces of the inhaler were rinsed with solvent including the interior of the capsule. The samples were then assayed using the UV-VIS spectrophotometer.

Shot weight was obtained by measuring the weight loss due to the actuation of the device. The device was tared, a “shot” was wasted in the DUSA and the device was re-weighed to obtain the delivered shot weight. Dose and shot weight are deemed acceptable if they are within 75% to 125% of the theoretical values (USP <601>).

Dose uniformity results for all blends are summarized in Table 3.

TABLE 3
Dose uniformity results for formulations I and III

| | Drug | | | | | | | | |
|---|---|---|---|---|---|---|---|---|---|
| | load | | Flow | | | Shot | Amount of Montelukast | | |
| | (% | Device | Rate | Duration | Formulation/ | weight | Acid recovered (mg) | | |
| Carrier | w/w) | Type | (L/min) | (Sec.) | Capsule # | (mg) | DUSA | Inhaler | Total |
| Lactose | 4 | Spinhaler | 98.1 | 2.5 | I/A | 24.4 | 0.32 | 0.37 | 0.70 |
| for | | | 77.2 | 3.1 | I/B | 25.2 | 0.45 | 0.44 | 0.89 |
| inhalation | 4 | Handihaler | 53.4 | 4.5 | I/C | 21.4 | 0.44 | 0.51 | 0.95 |
| | | | 55.2 | 4.3 | I/D | 25.8 | 0.65 | 0.23 | 0.88 |
| | 20 | Spinhaler | 71.9 | 3.3 | III/A | 23.2 | 1.82 | 1.52 | 3.34 |
| | | | 65.6 | 3.7 | III/C | 16.7 | 1.91 | 2.86 | 4.78 |

Table 3 shows that the shot weights for both 4% w/w blends were on target while the shot weight for capsule C for the 20% w/w blend were outside 75% and 125% of the theoretical values. During the collection of the drug from the DUSA and the DPI inhaler, it was observed that a fraction of powder remained in the capsule for the 20% w/w blend. The low shot weight and the powder remaining in the capsule may be explained by the fact that the 20% w/w blend contained more drug than the 4% w/w blend. This may have led to poor flow properties of the higher drug load formulation. This explanation can be supported by the morphological observation for the 20% w/w blend in which the drug has tendency to agglomerate and to create an interaction between the drug and the surface of the lactose, as discussed above. The average shot weights measured for the 4% w/w blend for capsules A and B performed with Spinhaler®, and capsules C and D performed with Handihaler® were 24.8 mg and 23.6 mg, respectively compared to 20.0 mg for the 20% w/w blend for capsules A and C performed with Spinhaler®.

The average amount of drug measured in the DUSA for capsules A and B, and C and D for 4% w/w blend were 38.5% and 54.5% of the nominal dose, respectively. The data also show that the drug amount that was expelled from the capsule was higher with Handihaler® than that noted for Spinhaler®. For the 20% w/w blend, the mass of drug recovered by percentage, 37.3%, in the DUSA was close to that observed for 4% w/w blend.

### Aerodynamic Particle Size Distribution

The Andersen cascade impaction (ACI) (Apparatus 3) was the device used to determine the aerodynamic size distribution. The impaction provided in vitro measurements of the fraction of the aerosol that has the potential to reach the alveolar region of the lung. This value is represented by the portion of particles detected below plate 2. The impaction was operated at the flow rate and test time according to the method described in USP <601>. Because the Spinhaler® is a low resistance device, it is difficult to achieve a pressure drop of 4 kPa, an adjustment of the air flow rate at the intake of the flow control was performed, as discussed above. Each impaction plate was coated with silicone grease (316 Dow Corning) to prevent particles from bouncing off the plates and returning to the air stream. All stages were used since the test flow rate was less than 60 L/min. All pieces of the impaction including the inhaler and capsule were rinsed with solvent, diluted to suitable volumes, sonicated, centrifuged and assayed using the UV-VIS spectrophotometer. The respirable portion was quantified by the in vitro fine particle fraction and fine particle mass. Dose uniformity and cascade impaction tests were carried out at controlled temperature (20-25° C.) and humidity (50% RH).

The aerodynamic particle size distribution data for all three blends are shown in Table 4.

TABLE 4
Cascade impaction results

| | | | | | In vitro fine | | |
|---|---|---|---|---|---|---|---|
| Batch | Drug | Target | | | particale | | Emitted |
| size | load | dose wt | | Blend ID/ | fraction | Fine particle | Dose |
| (g) | (% w/w) | (mg) | Device | Capsule # | (%) | mass (mg) | (mg) |
| 1 | 4 | 1 | Handihaler | I/E | 30 | 0.17 | 0.55 |
| | | | | I/F | 30 | 0.11 | 0.37 |
| 10 | 4 | 1 | Spinhaler | I/G | 22 | 0.03 | 0.13 |
| | | | | I/H | 37 | 0.08 | 0.22 |
| 10 | 20 | 5 | Spinhaler | III/F | 77 | 0.05 | 0.06 |
| | | | | III/G | 28 | 0.53 | 1.91 |
| | | | | III/H | 32 | 0.78 | 2.47 |

The mean fine particle fraction found with the HandiHaler® and Spinhaler® was 30% and 29.5%, respectively, for the 4% w/w blend. For the 20% w/w blend, a mean fine particle fraction of 45.3% was obtained using the Spinhaler®. In addition, the mean fine particle mass for the 4% w/w blend performed with Handihaler® and Spinhaler® was 0.14±0.04 mg and 0.06±0.04 mg, respectively. A fine particle mass of 0.45±0.4 mg was obtained for the 20% w/w blend. The results demonstrate that the drug disperses to the greatest extent in a 4% w/w blend performed with the HandiHaler®. For the 20% w/w blend, the emitted dose for capsule III/F was very low, indicating that the powder is somehow not expelled effectively from the capsule. The capsule orientation was checked before the inhaler was discharged. Therefore, a third trial was initiated to verify the aerosol performance of the 20% w/w blend. The data obtained for capsule III/H confirmed that the fine particle fraction for the 20% w/w drug loading is almost equal to the 4% w/w blend when the ACI was performed using the Spinhaler®.

### Blend Characterization/Morphology

Scanning electron micrographs (SEM) of the lactose reveal that the lactose has a plate-like morphology with a particle size up to about 140 μm and no observed agglomerates. For the blend of 4% micronized montelukast acid with 96% lactose, small irregularly-shaped particles attributed to montelukast acid compound with a particle size up to about 10 μm were observed. These SEM micrographs show that the drug is widely spread among the lactose particles. For the blend of 20% micronized montelukast acid with 80% lactose, more drug particles were observed in the blends. The drug appears to have a tendency to agglomerate, and a fraction of the drug appears to accumulate on the surface of the lactose. This phenomenon is also observed for the blend 4% w/w, but the degree of agglomeration is less evident due to the lower drug loading.

### In Vivo Evaluation of Montelukast Acid DPI Formulation

The allergic sheep model was used to test the effect of inhaled montelukast acid against early asthmatic response (EAR), late asthmatic response (LAR) and airway hyper-reactivity (AHR) response to Ascaris challenge in allergic sheep. The compound was administered directly into the lungs using the Spinhaler DPI that was attached directly to an indwelling endotracheal tube. Capsules used in the Spinhaler contained a micronized blend of 20% drug/80% lactose, corresponding to approx. 5 mg of the active compound. The compound was administered as a single dose 30 minutes before Ascaris challenge. To optimize delivery, each Spinhaler actuation was synchronized with a series of aspiratory cycles.

Doses for inhalation were selected based on total IV doses administered in sheep studies that had been conducted. Administration of 3 or 9 capsules should achieve a total inhaled dose of approximately 0.1 mg/kg and 0.3 mg/kg, respectively. The purported dose delivered is an estimate based on an experimentally determined fine particle fraction efficiency of 30%. Plasma drug levels were measured at various time points throughout the study.

Initial experiments (n=2) were performed with 0.1 mg/kg of montelukast acid. This dose produced partial inhibition of the LAR and AHR but not the EAR. The second set of experiments (n=4) was performed with 0.3 mg/kg of montelukast acid. Marked inhibition of all three phases of the response was achieved. Results obtained are summarized in Table 5.

TABLE 5
Montelukast Acid in Ascaris-challenged Conscious Sheep.

| | EAR (%) | LAR (%) | AHR (%) |
|---|---|---|---|
| Dose | Inhibition | inhibition | inhibition |
| Approx. 0.3 mg/kg | 47 | 79 | 76 |
| (9 capsules) | | | |
| Approx. 0.1 mg/kg | 7 | 42 | 60 |
| (3 capsules) | | | |

### EXAMPLE 2

### Compound X

### Compound X Description

Three jet milled samples of Compound X observed using X-ray powder diffraction (XRPD) and thermogravimetry (TGA) that the jet milled samples had similar properties to the unmilled lots. The samples retained their crystalline form. By SEM, it was observed that the jet milled drug was smaller in particle size compared to the unmilled drug, while maintaining the needle-like morphology. Drug particle size ranged from ca. 2-25 μm in length and ca. 2 μm in width with agglomerates up to 50 μm in diameter. Only one of the jet milled lots was used for the described studies below. A side by side comparison of the unmilled drug and the jet milled drug is shown in Table 6.

TABLE 6
Particle size of unmilled and jet milled Compound X

| | Unmilled API | Milled API | |
|---|---|---|---|
| | Optical microscopy | Microtrac data* | Aerosizer data |
| Mean (microns) | 11 | 2.794 | 2.832 |
| SD (microns) | 12 | 0.639 | 2.246 |
| 95% (microns) | 37 | 4.577 | 7.832 |

*After sonication for 60 seconds

### Carrier Characterization

Three different grades of lactose were investigated as carriers for Compound X, The carriers studied were milled lactose for inhalation, sieved lactose for inhalation and granulated lactose for inhalation. Each carrier was characterized for geometric diameter using an Aerosizer® LD and morphology using a JSM-5900LV scanning electron microscope; to assess carrier flow behavior, Carr's index was also obtained. The results are summarized in Table 7.

TABLE 7
Mean particle size and flow properties of various carriers

| | Geometric diameter (μm) | | Carr's |
|---|---|---|---|
| Excipient | Mean size | Std. dev. | index (%) Mean |
| Milled lactose | 35 | 1.5 | 52 |
| Sieved lactose | 41 | 1.4 | 31 |
| Granulated lactose | 59 | 1.6 | 35 |

From SEM micrographs, it was observed that granulated lactose had more surface porosity than milled or sieved lactose. Needle-like particles were observed for the micronized drug, which were similar to the unmilled GMP lots.

### Formulation

All blends were prepared in the same manner by blending in a low shear tumbling blender (Turbula Type T2F) for 15 minutes at 32 rpm. The blends contained 4% API and were manufactured at a scale of 1 g in a 4 ml glass amber bottle (50% fill volume). Then, 25 mg of blend, equivalent to 1 mg of drug, was weighed into each capsule (capsule size: 2LLC white opaque). The formulations are described in Table 8.

TABLE 8
DPI formulations with 4% drug loading and different carriers

| | | Formulation | | | |
|---|---|---|---|---|---|
| | | A | | | |
| | | (% | B | C | Drug only |
| Ingredient | Function | w/w) | (% w/w) | (% w/w) | (% w/w) |
| Milled lactose | Carrier | 96 | — | — | — |
| Sieved lactose | | — | 96 | — | — |
| Granulated | | — | — | 96 | — |
| lactose | | | | | |
| Compound X | API | 4 | 4 | 4 | 100 |
| Batch size (g) | — | 1 | 1 | 1 | — |
| Shot weight (mg) | — | 25 | 25 | 25 | 5 |
| Dose (mg) | — | 1 | 1 | 1 | 5 |

### Blend Uniformity

To assess blend uniformity, two capsules from each blend were opened, rinsed with solvent and assayed using a UV-Vis spectrophotometer. The solvent used for the DPI studies was a 60:40 mixture of methanol and water. The solvent was prepared in batches of 1000 ml. Six hundred milliliters of methanol was added to four hundred milliliters of water. The solution was then covered and allowed to cool to room temperature. To detect Compound X a calibration curve was developed using a UV-Vis spectrophotometer. In the 200 to 400 nm range, the maximum absorbance of Compound X was found to be 257 nm.

Blend uniformity results for formulations A, B and C are summarized in Table 9. It was observed that the amount of drug recovered was low for all blends. In addition, drug recovery in capsules A and B was considerably higher then C. The variable and low recovery may be due to poor blend uniformity and/or segregation during sampling and handling. Capsules with 5 mg of drug only were also prepared to observe the behavior of Compound X in the Spinhaler without the aid of a carrier (Table 9).

TABLE 9
Individual capsule assay for formulations A, B and C

| | | | | Mass of Compound |
|---|---|---|---|---|
| | Drug load | Batch size | Capsule | X recovered in |
| Formulation | (% w/w) | (g) | number | capsule (mg) |
| A | 4 | 1 | 1 | 0.79 |
| | | | 2 | 0.90 |
| B | 4 | 1 | 1 | 0.87 |
| | | | 2 | 0.77 |
| C | 4 | 1 | 1 | 0.26 |
| | | | 2 | 0.28 |

### Dose Uniformity Studies

Dose uniformity was determined using Apparatus B (Dosage Unit Sampling Apparatus—DUSA) at a flow rate of 100 L/min (test described in United States Pharmacopoeia (USP) 27 Chapter <601>). The USP recommends selecting a flow rate that creates a pressure drop of 4 kPa across the inhaler. With the Spinhaler, a 4 kPa pressure drop could not be achieved even at the maximum flow rate of 100 L/min. Based on the recommendations of Byron, et al., a flow rate of 100 L/min was selected since the Spinhaler is a low resistance device. See Michael Hindle and Peter R. Byron, “Dose emissions from marketed dry powder inhalers”, International Journal of Pharmaceutics 116 (1995) 169-177. The test was run for 2.4 seconds in order to pull 4 L of air. After a shot had been delivered, all pieces of the DUSA including the mouthpiece adapter were rinsed with solvent. To determine the amount of drug retained in the inhaler, all pieces of the inhaler were rinsed with solvent including the interior of the capsule. The samples were then assayed using the UV-Vis spectrophotometer.

Shot weight was obtained by measuring the weight loss due to the actuation of the device. The device was tared, a “shot” was wasted in the DUSA and the device was re-weighed to obtain the delivered shot weight. Dose and shot weight were deemed acceptable if they were within 75% to 125% of the theoretical values (USP <601>).

Dose uniformity results for formulations A, B and C are summarized in Table 10. It was observed that formulations B and C were on target for shot weight; however, formulation A was at or below the lower limit for acceptable shot weight, which may be attributed to the poor flow properties of the milled lactose. The average shot weights measured for B and C were 24.6±0.1 mg and 24.6±0.5 mg, respectively compared to 17.4±2.8 mg for A.

TABLE 10
Dose uniformity results for formulations A, B and C

| | | Drug | | Shot | Amount of Compound X | | |
|---|---|---|---|---|---|---|---|
| | | load | Trial | weight | recovered (mg) | | |
| Formulation | Carrier | (% w/w) | no. | (mg) | DUSA | Inhaler | Total |
| A | Milled lactose | 4 | 1 | 19.1 | 0.24 | 0.50 | 0.74 |
| | | | 2 | 14.2 | 0.22 | 0.98 | 1.19 |
| | | | 3 | 18.9 | 0.23 | 0.67 | 0.90 |
| B | Sieved lactose | 4 | 1 | 24.5 | 0.24 | 0.38 | 0.62 |
| | | | 2 | 24.6 | 0.32 | 0.66 | 0.98 |
| C | Granulated | 4 | 1 | 24.9 | 0.16 | 0.06 | 0.22 |
| | Lactose | | 2 | 24.2 | 0.15 | 0.09 | 0.24 |
| Drug only | — | 100 | 1 | 0.7 | 1.01 | 3.27 | 4.28 |
| | | | 2 | 0.6 | 0.91 | 3.36 | 4.27 |

For all formulations, dose weight was well below the target value of 1 mg. The average amount of drug measured in the DUSA for formulations A, B and C was 23%, 28% and 16% of the nominal dose, respectively. For formulation C, the low mass of drug recovered in the DUSA was probably due to the 23% total drug recovery as a result of blend uniformity issues. To remove the effect of blend uniformity, the emitted dose of formulations A, B and C will be compared in terms of the amount of drug measured in the DUSA divided by the total amount of drug recovered in the system (DUSA+inhaler). Therefore, the average amount of drug measured in the DUSA for formulations A, B and C was 25%, 36% and 68% of the total recovered dose, respectively. With only drug and no carrier, approximately 23% of the 5 mg nominal dose was recovered in the DUSA, which demonstrates the poor flowability of the drug in the Spinhaler. Only formulations B and C improved the flow of drug particles out of the inhaler as seen by the increased emitted doses. The emitted dose was considerably higher in formulation C. One possible explanation is that granulated lactose (formulation C) possessed a much more porous surface than milled lactose (formulation A) and sieved lactose (formulation B) resulting in stronger interparticulate bonds due to the entrapment of the fine drug particles within the surface cracks and dimples. The stronger interparticle interactions formed with granulated lactose allowed more drugs to be drawn out of the capsule with the carrier leaving fewer drugs behind in the inhaler. The surfaces of milled lactose (formulation A) and sieved lactose (formulation B) were smoother making it more difficult for the drug to interact with lactose. In addition to the surface properties of milled lactose, the poor flow properties of the carrier may have contributed to the low emitted dose observed in formulation A.

### Aerodynamic Particle Size Distribution

The Andersen cascade impactor (Apparatus 3) was the device used to determine the aerodynamic size distribution. The impactor provided in vitro measurements of the fraction of the aerosol that has the potential to reach the alveolar region of the lung. This value is represented by the portion of particles below plate 2. The impactor was operated at 100 L/min for 2.4 seconds according to the method described in USP 27 <601>. Each impactor plate was coated with silicone grease (316 Dow Corning) to prevent particles from bouncing off the plates and returning to the air stream. Plates 6 and 7 were omitted since the test flow rate was greater than 60 L/min. All pieces of the impactor including the inhaler and capsule were rinsed with solvent and assayed using the UV-Vis spectrophotometer. The respirable portion was quantified by the in vitro fine particle fraction and fine particle mass. Dose uniformity and cascade impaction tests were carried out at controlled temperature (20-25° C.) and humidity (35% RH).

The aerodynamic particle size distribution data for formulations A, B and C are shown in Table 11. The mean fine particle fraction was 54%, 30% and 9% for formulations A, B and C, respectively. In addition, the mean fine particle mass was 0.18±0.06 mg, 0.14±0.04 mg and 0.02±0.01 mg for A, B and C, respectively. The results demonstrate that the drug disperses to the greatest extent in formulation A and the least in formulation C. As mentioned previously, the results can be explained by the greater interparticle interactions formed in formulation C due to the higher surface porosity.

With only 5 mg of drug and no carrier the greatest respirable portion was achieved with a fine particle fraction of 65% and a mean fine particle mass of 0.62±0.04 mg.

TABLE 11
Cascade impaction results for formulations A, B and C

| | | Drug | | In vitro fine | Fine | |
|---|---|---|---|---|---|---|
| | Batch | load | Target dose | particle | particle | Emitted |
| Formulation | size (g) | (% w/w) | weight (mg) | fraction (%) | mass (mg) | Dose (mg) |
| A | 1 | 4 | 1 | 57 | 0.22 | 0.38 |
| | | | | 51 | 0.14 | 0.26 |
| B | 1 | 4 | 1 | 33 | 0.18 | 0.56 |
| | | | | 31 | 0.10 | 0.31 |
| | | | | 25 | 0.13 | 0.52 |
| C | 1 | 4 | 1 | 13 | 0.02 | 0.19 |
| | | | | 5 | 0.01 | 0.25 |
| Drug only | — | 100 | 1 | 62 | 0.59 | 0.95 |
| | | | | 68 | 0.64 | 0.95 |

Investigation into a Blend De-Lumping Step

In an attempt to improve blend uniformity an investigation into a blend de-lumping step was carried out. Two different de-lumping methods were considered for this study: milling and geometric dilution. Blend de-lumping was investigated with sieved lactose at different batch sizes (1 g and 25 g) and drug loads (4% w/w and 10% w/w). The processing conditions are outlined in Table 12.

TABLE 12
Formulations to investigate a blend de-lumping step

| | D | E | F | G |
|---|---|---|---|---|
| Ingredient | (% w/w) | (% w/w) | (% w/w) | (% w/w) |
| Sieved lactose | 96 | 96 | 96 | 90 |
| Compound X | 4 | 4 | 4 | 10 |
| Batch size (g) | 1 | 25 | 25 | 25 |
| Shot weight (mg) | 25 | 25 | 25 | 10 |
| Dose (mg) | 1 | 1 | 1 | 1 |
| De-lumping method | Milling | Geometric | Milling | Milling |
| | | dilution | | |
| Final mixing time | 2 | 6 | 1 | 1 |
| (min) | | | | |

Blends D (4% API), F (4% API) and G (10% API) were de-lumped using a milling step at a scale of 1 g, 25 g and 25 g, respectively. First, sieved lactose and Compound X were added to a 4 ml or 4 oz glass amber bottle (depending on the batch size) in order to achieve approximately 50% fill volume. The blends were then mixed in a low shear tumbling blender mixer for 15 minutes at 32 rpm. The blends were passed through a comill using a 0.016″ flat screen and square impeller at 29 rpm. The de-lumped blend was then blended in the mixer at 32 rpm for a duration of 1 to 2 minutes. For the 4% formulations, 25 mg of blend was weighed into each capsule in order to achieve 1 mg of drug per capsule. For the 10% formulation, 10 mg of blend was weighed into each capsule.

Formulation E (4% API) was prepared using a geometric dilution step at a scale of 25 g. The drug was sandwiched between two layers of lactose and carefully triturated in a mortar and pestle using low shear force. The contents of the mortar was emptied into a 4 oz. glass amber bottle and mixed in a mixer for 6 minutes at 32 rpm. Then, 25 mg of blend, equivalent to 1 mg of drug, was weighed into each capsule.

To assess blend uniformity, two capsules from each blend were opened, rinsed with solvent and assayed using a UV-Vis spectrophotometer. The aerodynamic particle size was also determined.

The results of these approaches are summarized in Table 13. It was observed that all blends were uniform; however, drug recovery was low for formulation 104 which may be due to scaling. One gram of blend was too small for the comill, which resulted in high material loss (24% of the blend was lost due to milling). Increasing the batch size improved drug recovery. At a 25-g scale, both milling and geometric dilution improved blend uniformity.

TABLE 13
Individual capsule assay for formulations B, D, E, F and G

| | | | | | Mass of |
|---|---|---|---|---|---|
| | Drug | | Additional | | Compound X |
| | load | Batch | processing | Capsule | in capsule |
| Formulation | (% w/w) | size (g) | steps | number | (mg) |
| B | 4 | 1 | — | 1 | 0.87 |
| | | | | 2 | 0.77 |
| D | 4 | 1 | Milling | 1 | 0.49 |
| | | | | 2 | 0.53 |
| E | 4 | 25 | Geometric | 1 | 1.09 |
| | | | dilution | 2 | 1.10 |
| F | 4 | 25 | Milling | 1 | 1.08 |
| | | | | 2 | 1.08 |
| G | 10 | 25 | Milling | 1 | 1.09 |
| | | | | 2 | 1.08 |

### Dose Uniformity Studies

Dose uniformity results are summarized in Table 14. It was observed that all formulations were within 75 to 125% of the target shot weight. Average shot weights for the 4% w/w blends 104, 114 and 122 were 22.9±1.1 mg, 24.0±0.4 mg and 23.1±0.7 mg, respectively. Shot weight was slightly lower for the 10% formulation at 85% of the target value. This result may be due to the poorer flow properties of the higher drug load formulation. Other studies on Compound X demonstrated that flow properties decreased as drug load increased.

For all formulations, dose weight was outside the acceptable limit of 75% to 125% of the nominal dose. Dose recovery in the DUSA was similar to formulation B for all blends. The emitted dose was slightly higher for formulation E. One possible explanation is that stronger interparticle interactions were formed between the drug and carrier during trituration. The stronger adhesion would allow more drug to leave the inhaler with the carrier.

TABLE 14
Dose uniformity results for formulations D, E, F and G

| | | | Add'l | Shot | Amount of Compound | | |
|---|---|---|---|---|---|---|---|
| | Drug load | Batch size | processing | weight | X recovered (mg) | | |
| Formulation | (% w/w) | (g) | steps | (mg) | DUSA | Inhaler | Total |
| B | 4 | 1 | — | 24.5 | 0.24 | 0.38 | 0.62 |
| | | | | 24.6 | 0.32 | 0.66 | 0.98 |
| D | 4 | 1 | Milling | 23.6 | 0.24 | 0.19 | 0.43 |
| | | | | 22.1 | 0.19 | 0.27 | 0.46 |
| E | 4 | 25 | Geometric | 24.2 | 0.40 | 0.41 | 0.82 |
| | | | dilution | 23.7 | 0.38 | 0.41 | 0.79 |
| F | 4 | 25 | Milling | 22.6 | 0.31 | 0.56 | 0.87 |
| | | | | 23.6 | 0.37 | 0.50 | 0.87 |
| G | 10 | 25 | Milling | 8.5 | 0.21 | 0.57 | 0.78 |
| | | | | 8.4 | 0.25 | 0.60 | 0.85 |

### Aerodynamic Particle Size Distribution

Aerodynamic particle size data generated by the Andersen cascade impactor is presented in Table 15. It was observed that introducing a blend de-lumping step, both milling and geometric dilution, decreased the respirable portion. This result may be explained by the greater drug/carrier interparticle interactions created as a result of milling and/or geometric dilution. Drug dispersion was lower with geometric dilution compared to milling. As mentioned previously, this result may be explained by the greater shear force exerted on the particles during trituration, which caused the drug to adhere more to the carrier particles.

TABLE 15
Cascade impaction results for formulations D, E, F and G

| | | | | | In vitro | |
|---|---|---|---|---|---|---|
| | | | | | fine | Fine |
| | | Add'l | Batch | Drug | particle | particle |
| | | processing | size | load | fraction | mass |
| Formulation | Carrier | steps | (g) | (% w/w) | (%) | (mg) |
| B | Sieved | — | 1 | 4 | 33 | 0.18 |
| | lactose | | | | 31 | 0.10 |
| | | | | | 25 | 0.13 |
| D | Sieved | Milling | 1 | 4 | 28 | 0.09 |
| | lactose | | | | 27 | 0.08 |
| E | Sieved | Geometric | 25 | 4 | 14 | 0.06 |
| | lactose | dilution | | | 15 | 0.06 |
| F | Sieved | Milling | 25 | 4 | 22 | 0.09 |
| | lactose | | | | 30 | 0.09 |
| G | Sieved | Milling | 25 | 10 | 21 | 0.08 |
| | lactose | | | | 19 | 0.09 |
| | | | | | 23 | 0.08 |

### Conclusion

An investigation into the aerosol performance of Compound X with different grades of lactose at 4% w/w drug loading demonstrated that sieved lactose was the most suitable carrier of the three choices. Granulated lactose produced the weakest drug aerosolization compared to milled and sieved lactose. Drug dispersion was the best with milled lactose; however, the poor flow properties of the carrier resulted in variable shot weight. Sieved lactose was chosen since the fine particle mass was similar to milled lactose and better shot weight was achieved with sieved lactose. Blend uniformity issues were encountered with all carriers. The introduction of a blend de-lumping step improved blend uniformity, but decreased the respirable portion.

A 4% w/w drug load formulation in sieved lactose with a milling step during blend preparation was found to possess a combination of superior properties. The delivered shot weight was 92% of target with an in-vitro fine particle fraction of 26% and an emitted dose of 34%.

### EXAMPLE 3

### Montelukast Acid and Compound X

The following formulations of montelukast acid and compound X may be prepared in accordance with the methods described in the previous examples:

| | | Formulation | | |
|---|---|---|---|---|
| Ingredient | Function | 4% w/w | 4% w/w | 20% w/w |
| Lactose for inhalation | Carrier | 92 | 92 | 76 |
| Montelukast Acid | API | 4 | 4 | 20 |
| Compound X | API | 4 | 4 | 4 |
| Batch size (g) | — | 1 | 10 | 10 |
| Shot weight (mg) | — | 25 | 25 | 25 |
| Capsule size | — | 2 | 2 | 2 |
| Dose (mg) | — | 1 | 1 | 5 |

Both montelukast acid and Compound X are shown to be moisture sensitive and photosensitive. A selection of the capsule and the package components for this combination formulation should take into account moisture and light protection, as well as the addition of a desiccant.

### EXAMPLE 4

### Montelukast Acid and Mometasone Furoate

### Preparation of DPI Formulation

Pre-blend Preparation: Magnesium stearate (MgSt) was first sieved through a 300 μm aperture sieve and then blended with lactose for inhalation in a mortar with pestle.
Formulation Preparation: mometasone furoate was transferred to the mortar and then was gently blended with the pre-blended lactose and MgSt with a pestle. This ternary blend was again blended with montelukast acid in the mortar and then with the remaining pre-blended lactose and MgSt. The final blend was sieved through a 300 μm aperture sieve before transferring to an ambler glass vial for blending in a Turbula tumbling mixer (Type T2F) for 10 minutes at 32 rpm.
   The formulation composition is shown in Table 16.A

TABLE 16.A
Formulation Composition

| | | Formulation |
|---|---|---|
| Ingredient | Function | % w/w |
| Lactose for inhalation | Carrier | 94.15 |
| Magnesium Stearate | Force Control Agent | 0.25 |
| Montelukast Acid | API | 4 |
| Mometasone Furoate | API | 1.6 |
| Batch size (g) | — | 1 |
| Shot weight (mg) | — | 25 |
| Capsule size | — | 2 |
| Dose (mg) | | |
| Montelukast Acid | API | 1 |
| Mometasone Furoate | API | 0.400 |

### Blend Uniformity

To assess blend uniformity, the blend was sampled randomly from the glass vial. The drugs were extracted analogous to that described in section Blend Uniformity for Example 1. However, the content of montelukast acid and mometasone furoate were analyzed by High Performance Liquid Chromatography (HPLC) employing a phenyl column with a controlled temperature of 50° C., a mixture of water containing 0.2% trifluoroacetic acid (TFA) and acetonitrile containing 0.2% TFA (53:47) as the mobile phase at a flow rate 2 ml/min and UV-detection at 248 nm.

The blend uniformity results are summarized in Table 16.B. The results show that the blend was uniform with the amount of drug content within ±10% of the nominal doses.

TABLE 16.B
Blend Uniformity Results

| | Amount Recovered (μg/Capsule) | | | |
|---|---|---|---|---|
| | Montelukast Acid | | Mometasone Furoate | |
| Capsule # | μg | % | μg | % |
| 1 | 940.4 | 91.4 | 386.8 | 93.9 |
| 2 | 907.0 | 93.0 | 371.6 | 93.5 |
| 3 | 953.9 | 93.9 | 389.5 | 95.8 |
| Mean ± RSD | 933.8 ± 2.6 | 92.8 ± 1.4 | 382.6 ± 2.6 | 94.4 ± 1.4 |

### Dose Uniformity

Dose Uniformity (DU) was performed according to USP Chapter <601> by using DUSA Apparatus B with the Spinhaler® device which is analogous to that described in section Dose Uniformity for Example 1. However, the HPLC was used to analyze the content of the drugs as described in the Blend Uniformity in this example.

Dose uniformity results are summarized in Table 16.C. The results show that the Spinhaler® gave a dose uniform of 49.3% and 55.5% based on the nominal dose for montelukast acid and mometasone furoate, respectively. The obtained dose uniformity with the low resistance Spinhaler® device are considered acceptable and comparable to the dose uniformity reported for the marked product which ranges from 60% to 100%.

TABLE 16.C
Dose Uniformity Results (MON = montelukast acid; MOM = mometasone furoate);
flow rate and test duration for capsules 1, 2 and 3 are Q = 62.3 L/min, T = 3.9 sec.; Q = 62.8 L/min,
T = 3.8 sec.; and Q = 61.5 L/min, T = 3.9 sec.; respectively)

| | | Device + | | | | Adapter + | | Total | |
|---|---|---|---|---|---|---|---|---|---|
| | | Capsule | | DUSA Body | | Filter | | Recovered | |
| | Delivered | MON, | MOM, | MON, | MOM, | MON, | MOM, | MON, | MOM, |
| Capsule # | Shot, mg | μg | μg | μg | μg | μg | μg | μg | μg |
| 1 | 23.62 | 462.40 | 165.97 | 418.93 | 188.81 | 101.89 | 53.96 | 983.22 | 408.74 |
| | | 459.39 | 165.37 | 418.04 | 188.15 | 101.73 | 53.70 | 979.15 | 407.23 |
| 2 | 25.59 | 468.24 | 187.90 | 121.87 | 62.29 | 347.80 | 143.96 | 937.91 | 394.15 |
| | | 473.48 | 189.44 | 125.49 | 62.46 | 350.55 | 144.84 | 949.52 | 396.73 |
| 3 | 23.32 | 465.70 | 179.94 | 112.15 | 59.03 | 374.21 | 158.07 | 952.06 | 397.04 |
| | | 463.98 | 178.36 | 113.39 | 59.40 | 371.63 | 157.01 | 949.00 | 394.77 |
| | Mean | 465.53 | 177.83 | 218.31 | 103.36 | 274.64 | 118.59 | 958.48 | 399.78 |
| | SD | 4.9 | 10.4 | 155.1 | 66.0 | 134.3 | 50.5 | 18.3 | 6.5 |
| | | *46.6 | 44.5 | | | 49.3 | 55.5 | | |
| | | **48.6 | 44.5 | | | 51.4 | 55.5 | | |

*DU based on nominal dose, %;
**DU based on total recovered, %

### Aerodynamic Particle Size Distribution.

Aerodynamic size distribution was performed according to USP Chapter <601> by using ACI Apparatus 3 with the Spinhaler® device analogous to that described in section Aerodynamic Particle Size Distribution for Example 1. However, the HPLC was used to analyze the content of the drugs as described in the Blend Uniformity in this example. The aerodynamic particle size distribution results are shown in Table 16.D, 16.D.A and 16.D.B.

Table 16.D.A and 16.D.B show that the Spinhaler® gave a FPF of 29% with a mean mass median aerodynamic diameter (MMAD) of 4.5 μm for montelukast acid and a FPF of 22% and a MMAD 4.0 μm for mometasone furoate. The obtained FPF with the low resistance Spinhaler® device are considered acceptable and comparable to the dose uniformity reported for the marked product which ranges from 20% to 30%.

TABLE 16.D
ACI Reading

| | | Air | | | | | | | |
|---|---|---|---|---|---|---|---|---|---|
| | | flow | Duration | | | | | | Delivered |
| | | read by | of test, | | | | Device + Capsule | | Shot |
| | | flow | 240/Q | | | | Before | After | Weight, |
| Capsule # | RH | meter, Q | Sec. | P2 | P3 | P3/P2 | discharged, g | Discharged, g | mg |
| I | ~55% | 52.9 | 4.5 | 56.3 | 27.7 | 0.49 | 14.3660 | 14.3440 | 22.00 |
| J | ~53% | 52.4 | 4.6 | 56.4 | 27.9 | 0.49 | 14.3972 | 14.3752 | 22.00 |
| K | ~53% | 53.3 | 4.5 | 56.9 | 27.6 | 0.49 | 14.3716 | 14.3521 | 19.50 |

TABLE 16.D.A
ACI Results for Montelukast Acid (MON)

| | | MON | Total | | | | |
|---|---|---|---|---|---|---|---|
| | MON in | in | MON | | | | |
| | inhaler, | ACI, | recovered, | FPD, | FPF, | MMAD, | |
| Capsule # | μg | μg* | μg | μg | % | μm** | GSD |
| I | 450 | 513 | 962 | 145.7 | 28.4 | 4.5 | 1.4 |
| J | 524 | 397 | 921 | 118.7 | 29.9 | 4.4 | 1.4 |
| K | 512 | 441 | 953 | 126.1 | 28.6 | 4.6 | 2.1 |
| Mean | 495 | 450 | 945 | 130.2 | 29.0 | 4.5 | 1.6 |

*Including mouthpiece adapter
**Aerodynamic cutoff diameter is based on a volumetric airflow rate of 28.3 L/min

TABLE 16.D.B
ACI Results for Mometasone Furoate (MOM)

| | MOM | MOM | Total | | | | |
|---|---|---|---|---|---|---|---|
| | in | in | MOM | | | | |
| Capsule | inhaler, | ACI, | recovered, | FPD, | FPF, | MMAD, | |
| # | μg | μg* | μg | μg | % | μm** | GSD |
| I | 165 | 237 | 402 | 53.9 | 22.7 | 4.1 | 1.4 |
| J | 206 | 189 | 395 | 41.8 | 22.1 | 4.0 | 1.4 |
| K | 198 | 207 | 404 | 43.5 | 21.0 | 3.9 | 1.5 |
| Mean | 189 | 211 | 400 | 46.4 | 21.9 | 4.0 | 1.4 |

*Including mouthpiece adapter
**Aerodynamic cutoff diameter is based on a volumetric airflow rate of 28.3 L/min

### EXAMPLE 5

### Montelukast Acid and Ciclesonide

### Preparation of DPI Formulation

The formulation was prepared in a manner analogous to that described in Example 4, except mometasone furoate was replaced with ciclesonide and the excipients were adjusted accordingly. The final formulation composition is shown in Table 17.A.

TABLE 17.A
Formulation Composition

| | | Formulation |
|---|---|---|
| Ingredient | Function | % w/w |
| Lactose for inhalation | Carrier | 95.11 |
| Magnesium Stearate | Force Control Agent | 0.25 |
| Montelukast Acid | API | 4 |
| Ciclesonide | API | 0.64 |
| Batch size (g) | — | 1 |
| Shot weight (mg) | — | 25 |
| Capsule size | — | 2 |
| Dose (mg) | | |
| Montelukast Acid | API | 1 |
| Ciclesonide | API | 0.160 |

### Blend Uniformity

The blend uniformity was assessed in a manner analogous to that described in the Blend Uniformity section in Example 4, except the content of montelukast acid and ciclesonide were analyzed by High Performance Liquid Chromatography (HPLC) employing a phenyl column with a controlled temperature of 50° C., a mixture of water containing 0.2% trifluoroacetic acid (TFA) and acetonitrile containing 0.2% TFA (40:60) at a flow rate 2 ml/min and UV-detection at 248 nm.

The blend uniformity results are summarized in Table 17.B. The results show that the blend was uniform with the amount of drug content within ±10% of the nominal doses.

TABLE 17.B
Blend Uniformity Results

| | Amount Recovered (μg/Capsule) | | | |
|---|---|---|---|---|
| | Montelukast Acid | | Ciclesonide | |
| Capsule # | μg | % | μg | % |
| 1 | 943.3 | 94.3 | 150.9 | 93.3 |
| 2 | 928.8 | 92.9 | 146.7 | 93.7 |
| 3 | 997.5 | 99.8 | 157.6 | 92.5 |
| Mean ± RSD | 956.6 ± 3.4 | 95.7 ± 3.4 | 151.1 ± 3.3 | 93.2 ± 0.6 |

### Dose Uniformity

Dose Uniformity was performed according to USP Chapter <601> by using DUSA Apparatus B with the Spinhaler® device and in a manner analogous to that described in section Dose Uniformity in Example 4, except the HPLC was used to analyze the content of the drugs as described in the Blend Uniformity in this example.

Dose uniformity results are summarized in Table 17.C. The results show that the Spinhaler® gave a dose uniformity of 47.8% and 61.7% based on the nominal dose for montelukast acid and ciclesonide, respectively. The obtained dose uniformity with the low resistance Spinhaler® device is considered acceptable and comparable to the dose uniformity reported for the marked product which ranges from 60% to 100%.

TABLE 17.C
Dose Uniformity Results (MON = montelukast acid; CIC = ciclesonide); flow rate
and test duration for capsules A, B and C are Q = 59.8 L/min, T = 4.0 sec.;
Q = 58.1 L/min, T = 4.1 sec.; and Q = 59.7 L/min, T = 4.0 sec., respectively)

| | | Device + | | | | Adapter + | | Total | |
|---|---|---|---|---|---|---|---|---|---|
| | | Capsule | | DUSA Body | | Filter | | Recovered | |
| | Delivered | MON, | CIC, | MON, | CIC, | MON, | CIC, | MON, | CIC, |
| Capsule # | Shot, mg | μg | μg | μg | μg | μg | μg | μg | μg |
| A | 20.55 | 409.03 | 53.91 | 345.13 | 63.62 | 137.78 | 33.74 | 891.94 | 151.27 |
| | | 408.15 | 53.44 | 345.36 | 63.94 | 137.45 | 33.94 | 890.96 | 151.32 |
| B | 20.12 | 479.07 | 59.66 | 283.84 | 55.55 | 147.29 | 36.91 | 910.20 | 152.12 |
| | | 478.54 | 58.73 | 283.06 | 55.65 | 147.05 | 36.07 | 908.65 | 150.46 |
| C | 22.51 | 450.45 | 56.07 | 196.38 | 46.49 | 324.59 | 59.44 | 971.42 | 161.99 |
| | | 446.81 | 55.89 | 195.87 | 47.41 | 324.40 | 59.30 | 967.09 | 162.60 |
| | Mean | 445.34 | 56.28 | 274.94 | 55.44 | 203.09 | 43.23 | 923.38 | 154.96 |
| | SD | 31.5 | 2.5 | 67.0 | 7.5 | 94.1 | 12.6 | 36.5 | 5.7 |
| | | *44.5 | 35.2 | | | 47.8 | 61.7 | | |
| | | **48.2 | 36.3 | | | 51.8 | 63.7 | | |

*DU based on nominal dose, %;
**DU based on total recovered, %

### Aerodynamic Particle Size Distribution.

Aerodynamic size distribution was performed according to USP Chapter <601> by using ACI Apparatus 3 with the Spinhaler® device in a manner analogous to that described in section Aerodynamic Particle Size Distribution in Example 4. The content of the drugs were analyzed as described in the Blend Uniformity in this example. The aerodynamic particle size distribution results are shown in Tables 17.D, 17.D.A, 17.D.B.

Tables 17.D.A and 17.D.B show that the Spinhaler® gave a FPF of 38% with a mean mass median aerodynamic diameter (MMAD) of 3.9 μm for montelukast acid and a FPF of 31% and a MMAD 3.7 μm for ciclesonide. The obtained FPF with the low resistance Spinhaler® device are considered acceptable and comparable to the dose uniformity reported for the marked product which ranges from 20% to 30%.

TABLE 17.D
ACI Reading

| | | Air | | | | | | | |
|---|---|---|---|---|---|---|---|---|---|
| | | flow | Duration | | | | | | Delivered |
| | | read by | of test, | | | | Device + Capsule | | Shot |
| | | flow | 240/Q | | | | Before | After | Weight, |
| Capsule # | RH | meter, Q | Sec. | P2 | P3 | P3/P2 | discharged, g | Discharged, g | mg |
| E | ~55% | 51.9 | 4.6 | 59.9 | 27.0 | 0.45 | 14.3786 | 14.3562 | 22.36 |
| F | ~55% | 51.8 | 4.6 | 59.7 | 26.9 | 0.45 | 14.3803 | 14.3586 | 21.66 |
| G | ~55% | 52.4 | 4.6 | 60.0 | 27.0 | 0.45 | 14.3708 | 14.3488 | 21.91 |

TABLE 17.D.A
ACI Results for Montelukast Acid (MON)

| | | | Total | | | | |
|---|---|---|---|---|---|---|---|
| | MON in | MON in | MON | | | | |
| Capsule | inhaler, | ACI, | recovered, | FPD, | FPF, | MMAD, | |
| # | μg | μg* | μg | μg | % | μm** | GSD |
| E | 413 | 468 | 882 | 190.6 | 40.7 | 4.0 | 1.5 |
| F | 431 | 472 | 904 | 179.0 | 37.9 | 3.8 | 2.0 |
| G | 530 | 459 | 990 | 161.8 | 35.2 | 3.9 | 1.5 |
| Mean | 458 | 467 | 925 | 177.1 | 37.9 | 3.9 | 1.7 |

*Including mouthpiece adapter
**Aerodynamic cutoff diameter is based on a volumetric airflow rate of 28.3 L/min

TABLE 17.B.B
ACI Results for Ciclesonide (CIC)

| | | CIC | Total | | | | |
|---|---|---|---|---|---|---|---|
| | CIC | in | CIC | | | | |
| Capsule | inhaler, | ACI | recovered, | FPD, | | MMAD, | |
| # | μg | μg* | μg | μg | FPF, % | μm* | GSD |
| E | 53 | 107 | 160 | 31.1 | 29.1 | 3.8 | 1.5 |
| F | 56 | 122 | 178 | 41.4 | 33.9 | 3.8 | 1.6 |
| G | 67 | 110 | 177 | 33.6 | 30.6 | 3.6 | 1.7 |
| Mean | 59 | 113 | 172 | 35.5 | 31.2 | 3.7 | 1.6 |

*Including mouthpiece adapter
**Aerodynamic cutoff diameter is based on a volumetric airflow rate of 28.3 L/min

1. A medicinal preparation comprising montelukast acid and a second active agent selected from a PDE-4 inhibitor and an inhaled corticosteroid as a combined preparation for simultaneous, sequential or separate administration by inhalation.

2. A preparation of claim 1 wherein said second active agent is Compound X having the formula:

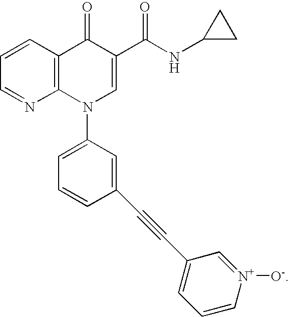

3. A preparation of claim 1 wherein said second active agent is mometasone furoate.

4. A preparation of claim 1 adapted for use in a dry powder inhaler or a metered dose inhaler.

5. A preparation of claim 2 adapted for use in a dry powder inhaler or a metered dose inhaler.

6. A preparation of claim 3 adapted for use in a dry powder inhaler or a metered dose inhaler.

7.-10. (canceled)

11. A method for the treatment of respiratory disorder comprising the simultaneous, sequential or separate administration by inhalation to a patient in need thereof a therapeutically effective amount of montelukast acid and a therapeutically effective amount of a second active agent selected from a PDE-4 inhibitor and an inhaled corticosteroid.

12. A method of claim 11 wherein said second active agent is Compound X.

13. A method of claim 11 wherein said second active agent is mometasone furoate.

14. A method of claim 11 wherein said second active agent is ciclesonide.

15. A method of claim 11 wherein said respiratory disorder is asthma.

16. A dry powder inhaler which contains the medicinal preparation of claim 1.

17. A metered dose inhaler which contains the medicinal preparation of claim 1.
